(19) United States
(12) Patent Application Publication
(10) Pub. No.: US 2011/0223251 A1
(43) Pub. Date: Sep. 15, 2011

(54) BACTERIAL COMPOSITION

(75) Inventors: Susanne Abrahamsen, Karlslunde (DK); Birgitte Stuer-Lauridsen, Virum (DK); Mette Winning, Soeborg (DK); Lasse Vigel Joergensen, Vaerlose (DK); Iben Jeppesen, Alleroed (DK); Peter Wagner, Hoersholm (DK)

(73) Assignee: CHR-HANSEN A/S

(21) Appl. No.: 13/059,614

(22) PCT Filed: Aug. 27, 2009
(86) PCT No.: PCT/EP2009/061085

§ 371 Date: May 4, 2011

(30) Foreign Application Priority Data
Aug. 28, 2008 (DK) PA 2008 01181
Dec. 2, 2008 (EP) 08170439.7

(51) Int. Cl. A61K 9/28; A61K 35/74; A61P 1/00

(52) U.S. Cl. 424/474; 424/93.4

(57) ABSTRACT

The present invention relates to a bacterial composition which has a long shelf-life, and a process for producing such compositions. The invention further relates to food products comprising the bacterial composition.

### FIELD OF INVENTION

The present invention relates to a bacterial composition which has a long shelf-life, and a method for producing such compositions. The invention further relates to food products comprising the bacterial composition.

### BACKGROUND OF INVENTION

Several patent documents disclose bacterial formulations which are formulated with specific excipient or carriers which are claimed to increase the storability of the bacterial formulations. Other patent documents relate to lowering the water activity (aw) of the bacterial formulation, e.g. by adding SiO2, which will bind some water. Also use of desiccant in different forms has been used to remove water.

U.S. Pat. No. 4,956,295 discloses dried Lactobacilli mixed with a silica gel absorbent. The mixtures are claimed to be storable without refrigeration.

U.S. Pat. No. 7,122,370B and U.S. Pat. No. 7,229,818B disclose a formulation comprising probiotic bacteria with monovalent alginate salts, wherein the formulation has a water activity of between 0.01 and 0.07, and wherein, upon exposure to an acidic environment, an alginic acid gel is formed which shields the probiotic bacteria from the antibiotic effects of the acidic environment. The alginate salt is dried to a moisture content below 5% before mixing with the bacteria.

EP1482811B discloses a process for obtaining a food product comprising the steps of

a. mixing a preparation of viable micro-organisms and further components,
b. drying the mixture to a water activity below 0.3,
c. compacting the mixture
d. coating, and
e. mixing with a food product.

US2005/0100559 discloses a dry bacterial composition having a water activity of less than 0.5.

U.S. Pat. No. 6,953,592B discloses a non-foaming, water-soluble or water-dispersible carbohydrate-based matrix containing entrapped gas in closed pores in an amount which is sufficient to promote dissolution or dispersion of the matrix upon contact with water.

US2005/0266069 claims a viable and stable probiotic formulation for intestinal targeting, comprising: a plurality of probiotic microspheres each comprising: a core comprising one or more probiotic bacteria, a cellulosic excipient, a disintegrant and one or more additives; and an enteric coating capable of being resistant to gastric fluids, having a residual moisture level of less than 5% and a water activity (aw) between 0.1 and 0.5.

WO 2005/063200 discloses a probiotic tablet comprising a probiotic micro-organism (e.g. Lactobacillus GG) and other nutritionally active ingredients in two zones; a first zone comprising said probiotic micro-organism and a second zone comprising at least one said other active ingredient. The water activity in the first zone should be no greater than 0.2. It is stated that good viability of the micro-organisms is obtained despite the relatively high overall moisture content.

WO08048731A claims a method for extending the shelf-life of an LGG-containing powdered nutritional formulation by reducing the water activity of the LGG-containing formulation to less than about 0.16 and maintaining the temperature of the formulation at or below 25 Degrees C.

U.S. Pat. No. 7,037,708B discloses a composition which comprises at least one Lactobacillus plantarum in carrier-bound form, which a) has a particle size of at least about 0.1 mm and b) comprises from about 10E10 to 10E12 cfu/g of at least one microorganism species; c) has a water activity (aw) of less than 0.15 and d) is compressed. It is disclosed that the culture may comprise an effervescent additive.

However, none of these bacterial formulations are satisfactory from a commercial point of view, especially for Bifidobacterial cells. Therefore, a need exists for improved bacterial cells containing compositions, having improved stability and increased delivery of viable bacterial cells.

In particular, a need exists for providing stable compositions comprising Bifidobacterial strains, which have previously been very difficult to store long-term at room temperature.

### SUMMARY OF INVENTION

The present inventors have surprisingly found out that a tablet which contains cells of a Bifidobacterium strain and a salt of carbonic acid, sodium bicarbonate, has superior stability especially if stored in an environment protecting against exposure to moisture from the surroundings, e.g. in a sealed aluminum bag or closure, or in a closed glass container. The superior stability is most pronounced when the protected tablets are exposed to high temperature over a longer period of time.

Further, the inventors have discovered that addition of a salt of carbonic acid to a composition containing cells of a Bifidobacterium strain surprisingly prevents or reduces the discoloration (red-coloring) that normally occurs during storage.

Based on these surprising findings, the present invention relates to a process for stabilizing (e.g. increasing the stability and/or reducing/avoiding the discoloration) of a composition comprising living cells of a Bifidobacterium strain by adding a salt of carbonic acid to the composition. Especially, the invention relates to a process for improving the color stability of a composition containing bacterial cells, said process comprises mixing the bacterial cells with a salt of carbonic acid.

The present invention can be seen as a process for improving the stability/shelf life of a bacterial composition, and thus the present invention also relates to a process for preparing a bacterial composition with improved increased/improved/prolonged stability/shelf life, which comprises the following steps:
a) providing a powder by mixing bacterial cells, preferably belonging to the genus Bifidobacterium, and a carrier comprising a salt of carbonic acid; and
b) optionally compressing the powder to form a tablet or a pellet.

The invention also relates to the stabilized compositions, and to a food product containing the stabilized composition, either integrated in the food product, or packaged together with the food product.

### DETAILED DISCLOSURE

In a first aspect, the present invention relates to a process for preparing a bacterial composition (e.g. for improving the survival of the cells and/or reduction of discoloration of the composition), which comprises the following steps:
a) providing a powder by mixing bacterial cells and a carrier comprising a salt of carbonic acid (or carbonate salt); and
b) optionally compressing the powder.

Further, the invention relates to processes for i) increasing the survival time of the bacterial cells in a composition, or ii) avoiding or reducing the discoloration of a bacterial composition, or iii) increasing the shelf life of a bacterial composition, or iv) increasing the stability of a bacterial composition, which processes comprise the following steps:
a) providing a powder by mixing bacterial cells and a carrier comprising a salt of carbonic acid (or carbonate salt); and
b) optionally compressing the powder.

In an interesting embodiment of the invention, the process is used for improving the stability (including the color stability) of a composition containing bacterial cells, by mixing the bacterial cells with a salt of carbonic acid.

It should be understood that the aim of the mixing step a) is to bring the bacterial cells in contact with the salt, and thus the sequence of mixing is not important. Thus, the bacterial cells may be mixed with a carrier before and/or after and/or at the same time as the mixing with the salt. It is presently preferred that all constituents are mixed at the same time.

By compressing the powder, the product of a process of the invention will be a bacterial composition in the form of a powder, or a solid form such as a tablet or a pellet.

The salt of carbonic acid is preferably selected from the group consisting of sodium carbonate, sodium bicarbonate, sodium sesquicarbonate, potassium carbonate, potassium bicarbonate, potassium sesquicarbonate, magnesium carbonate, sodium glycine carbonate, L-lysine carbonate, arginine carbonate, amorphous calcium carbonate, ammonium carbonate, ammonium bicarbonate and combinations thereof, and presently the most preferred salt is sodium bicarbonate.

The carrier in a) may further comprises an acidic component, such as an organic acid, preferably as a powder.

The acidic component is preferably selected from the group consisting of citric acid, tartaric acid, amalic acid, fumaric acid, adipic acid, lactic acid, succinic acid, disodium hydrogen phosphate, sodium dihydrogen phosphate, and combinations thereof.

In an embodiment of the invention, a process further may includes a drying step, such as freeze-drying, vacuum drying, drying by means of a desiccant, or heating. A drying step may be performed:

i) before the mixing step (i.e. one or more of the bacterial cells or carrier are dried); and/or
ii) after the mixing step (i.e. the mixture is dried); and/or
iii) after the optional compressing step (i.e. the tablet or pellet is dried).

The bacterial cells are preferably lactic acid producing and/or probiotic cells, such as bacterial cells belonging to a genus selected from the group comprising: Bifidobacterium, Lactobacillus, and Streptococcus. It is presently preferred that the bacterial cells belongs to a species selected from the group consisting of: Lactobacillus casei, Lactobacillus acidophilus, Lactobacillus reuteri, Lactobacillus bifidum, Lactobacillus gasseri, Lactobacillus plantarum, Lactobacillus johnsonii, Lactobacillus rhamnosus, Lactobacillus fermentum, Lactobacillus paracasei, Lactobacillus crispatus, Bifidobacterium longum, Bifidobacterium bifidum, Bifidobacterium lactis, Bifidobacterium brevis, Bifidobacterium animalis, Bifidobacterium adolescentis, Bifidobacterium infantis, Streptococcus thermophilus and Lactococcus lactis, such as bacterial cells belonging to a strain selected from the group consisting of: BB-12®, LA-1, LA-5, BB-02, Bifidobacterium animalis strain DSM15954, Bifidobacterium longum subsp. infantis strain DSM15953, Bifidobacterium longum subsp. longum strain DSM15955, Enterococcus faecium strain DSM15958, Lactobacillus acidophilus strain DSM13241, Lactobacillus delbrueckii subsp. bulgaricus strain DSM15956, Lactobacillus helveticus strain DSM14998, Lactobacillus helveticus strain DSM14997, Lactococcus lactis strain DSM14797, Streptococcus thermophilus strain DSM15957, Lactobacillus fermentum strain ATCC55845, and Lactobacillus rhamnosus strain ATCC55826, and mutants or variants of any of these. It is presently most preferred that the bacterial cells belong to the bifidobacterium strain BB-12® or BB-12® free.

The carrier may further comprise a component selected from the group consisting of: an inorganic acid or a salt thereof, an organic acid or a salt thereof, a carbohydrate, lactose, a sugar alcohol, soluble fibers and starch.

The processes of the invention may include further steps, such as a coating step (wherein e.g. a tablet/pellet of the invention is coated in a manner known to the skilled person), and/or a packaging step, e.g. comprising placing the powder or the compressed powder (tablet/pellet) in a sealable container, e.g. made of aluminum and/or a polymer. 2. The process of claim 1 for preparing a bacterial composition with no or reduced discoloration, which comprises the following steps:
a) providing a powder by mixing bacterial cells and a carrier comprising a salt of carbonic acid; and
b) optionally compressing the powder to form a tablet or a pellet.

3. The process of claim 1 for preparing a bacterial composition with improved survival of the cells and/or increased shelf life, and/or increased stability, which comprises the following steps:
a) providing a powder by mixing bacterial cells and a carrier comprising a salt of carbonic acid; and
b) optionally compressing the powder to form a tablet or a pellet.

4. The process of claim 1 for improving the survival of cells in a composition containing bacterial cells, comprising mixing the bacterial cells with a salt of carbonic acid.

5. The process of claim 1 for improving the color stability of a composition containing bacterial cells, comprising mixing the bacterial cells with a salt of carbonic acid.

In a second aspect, the invention relates to a composition obtainable by a process of the invention. A composition of the invention comprises bacterial cells and a carrier comprising a salt of carbonic acid (or a carbonate salt). The bacterial cells may belong to any of the above mentioned species or strains. In a presently interesting embodiment, the cells belong to the bifidobacterium strain BB-12®. The composition may comprise at least 10E5 CFU/mg, such as at least 10E7 CFU/mg or at least 10E9 CFU/mg, CFU being cell forming units of the bacterial cells.

In a third aspect, the present invention relates to the use of a composition of the present invention as a food, feed additive, a pharmaceutical, a dietary supplement, or a probiotic. A food or feed product comprising a composition of the invention is also an aspect of the present invention, and comprises products such as: a dairy product, such as milk or fermented milk, and a fruit juice, such as a smoothie.

The food/feed product may be mixed with the composition of the invention, either at the food/feed processing facility, or by the consumer. Thus, the present invention also relates to a kit of parts, comprising a food/feed product and a composition of the invention, e.g. a kit which comprises a container with a food product and container with a composition of the invention.

In an other aspect, the invention relates to the use of a salt of carbonic acid (e.g. salts as mentioned above) as i) stabilizer, such as a color stabilizer, ii) for increasing the shelf life, iii) for increasing the stability, or iv) for increasing the survival of cells, in a bacterial composition, especially a bacterial composition comprising bacterial cells belonging to the genus Bifidobacterium (e.g. belonging to a species selected from the group consisting of: Bifidobacterium longum, Bifidobacterium bifidum, Bifidobacterium lactis, Bifidobacterium brevis, Bifidobacterium animalis, Bifidobacterium adolescentis, Bifidobacterium infantis).

### DEFINITIONS

In the present context, the term “tablet” refers to a compressed powder. The term includes all physical forms and all sizes, such as a pill, a pellet, a tablet, etc.

By the term “bacterial composition” should be understood a composition comprising bacterial cells, or a cell culture. The cells is preferably living or dormant, and it is further preferred that the composition contains at least 10E5 cell forming units per gram. The bacterial cells may belong to a single strain, or be a mixture of cells belonging to different strains.

In the present context, the term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties as the mother strain, e.g. as a probiotic. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant.

In the present context, the term “variant” should be understood as a strain which is functionally equivalent to a strain of the invention, e.g. having substantially the same, or improved properties, e.g. as a probiotic. Such variants, which may be identified using appropriate screening techniques, are a part of the present invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein, is intended merely to better illuminate the invention and does not pose a limitation on the scope of the invention unless otherwise claimed. No language in the specification should be construed as indicating any non-claimed element as essential to the practice of the invention.

### LEGENDS

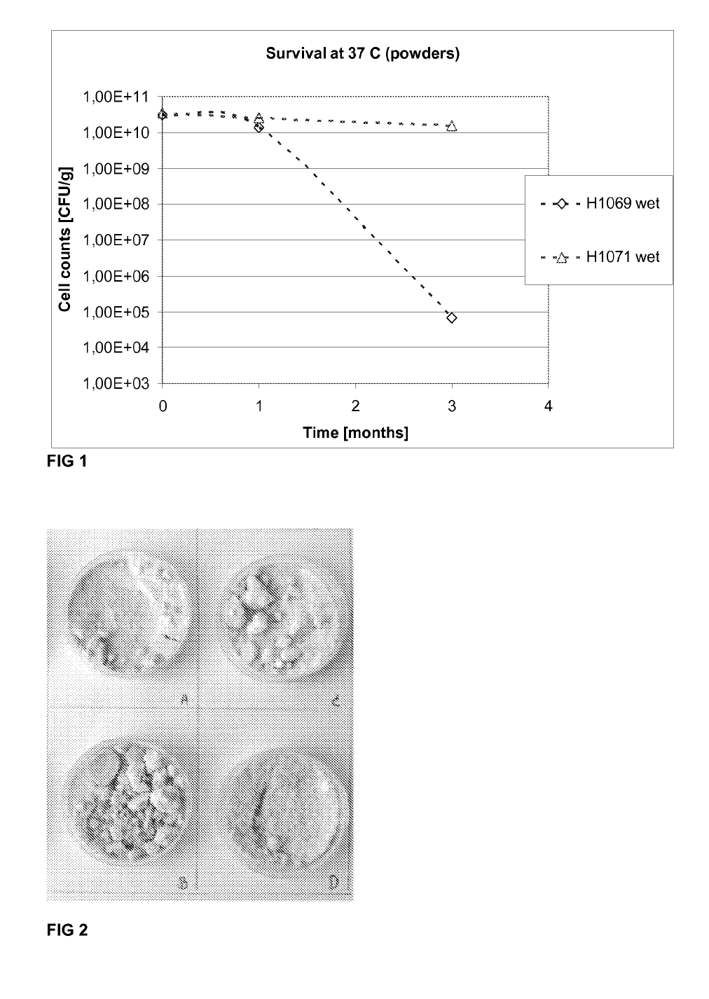

FIG. 1: Survival of BB-12® in powder H1069 and H1071 when stored at 37° C. for 3 months.

FIG. 2: Picture showing the powder blends H1184 A (A), H1184 B (B), H1184 C (C) and H1184 D (D) after two weeks at room temperature.

### EXAMPLES

### Example 1

Two blends are made, a powder blend H1069 containing grinded BB-12® HA (5.1% (w/w) and excipients (Mannitol; 10% (w/w), Lactose; 84.4% (w/w) Magnesium stearate; 0.5% (w/w) without Sodium bicarbonate, and powder blend H1071 contained grinded BB-12® HA (5.1% (w/w) and excipients (Mannitol; 10% (w/w), Lactose; 54.4% (w/w) Magnesium stearate; 0.5% (w/w) with Sodium bicarbonate; 30% (w/w). The concentration of Sodium bicarbonate is obtained by replacing the lactose part of the excipients with sodium bicarbonate leaving all other components at equal concentrations in the powder.

The powders were packed in aluminum foil bags (20-30 g) and stored at 37° C. for 3 months. Bags were taken for analysis of colony forming units (cfu/g) and water activity (aw) just after packaging (0 months), 1 month and 3 month of storage.

Results: The water activity of the powders H1069 and H1071 was equivalent, aw (H1069)=0.19 and aw (H1071)=0.19 just after packaging. After storage for 1 and 3 months the water activity of the powders increased to aw=0.27-0.31. The results of the colony forming units showed equivalent cfu/g at 0 and 1 month but showed a dramatic difference in survival of BB-12® after 3 months (FIG. 1).

Conclusion: The replacement of the dextrose excipient with sodium bicarbonate resulted in an improved survival of BB-12® during storage. This result shows that sodium bicarbonate improve survival of Bifidobacterium animalis in powders during storage.

### Example 2

### Test if Sodium Bicarbonate Will Influence the Red Color Development in Formulations of Bifidobacterium Containing Ascorbate

Contents of powder blends (w/w)
H1184 A: 24% grinded BB-12® HA culture, 51% mannitol & 25% inulin
H1184 B: 24% grinded BB-12® HA culture, 51% mannitol & 25% sodium bicarbonate
H1184 C: 12% grinded BB-12® HA culture, 63% mannitol & 25% inulin
H1184 D: 12% grinded BB-12® HA culture, 63% mannitol & 25% sodium bicarbonate

The grinded BB-12® HA culture contains ascorbate as a cryoprotective agent. The ascorbate reacts with residues from the fermentation and develops a red color. The reaction is accelerated by elevated temperatures and humidity.

The powders were kept in air-tight aluminum foil bags. For the testing the powders were taken out of the alu bags in amounts of 5-8 g and placed in small plastic containers (manufactured by Rotronic for use in aw measurements). The plastic containers were left at room temperature and after two weeks the difference in color development could be seen as shown in FIG. 2. The powders had a similar aw of approx. 0.4. FIG. 2 shows that the A and C powders have changed color to pale red, while the B and D powders have kept their original pale yellow color.

Conclusion: The presence of sodium bicarbonate in the powder blends has an effect on the development of red color. The red color does not develop when sodium bicarbonate is present in the powder blend.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

US2004175389, WO03075676, US2005100559, U.S. Pat. No. 6,953,592, WO03012819, WO05266069, WO05063200

All references cited in this patent document are hereby incorporated herein in their entirety by reference.

1-39. (canceled)

40. A process for preparing a bacterial composition, which comprises the following steps:
a) providing a powder by mixing bacterial cells belonging to the genus Bifidobacterium and a carrier comprising a salt of carbonic acid; and
b) optionally compressing the powder to form a tablet or a pellet.

41. The process of claim 40 for preparing a bacterial composition with no or reduced discoloration, which comprises the following steps:
a) providing a powder by mixing bacterial cells belonging to the genus Bifidobacterium and a carrier comprising a salt of carbonic acid; and
b) optionally compressing the powder to form a tablet or a pellet.

42. The process of claim 40 for preparing a bacterial composition with improved survival of the cells and/or increased shelf life, and/or increased stability, which comprises the following steps:
a) providing a powder by mixing bacterial cells belonging to the genus Bifidobacterium and a carrier comprising a salt of carbonic acid; and
b) optionally compressing the powder to form a tablet or a pellet.

43. The process of claim 40, wherein the bacterial composition is in the form of a powder, a granulate, a tablet or a pellet.

44. The process of claim 40, wherein the salt of carbonic acid is selected from the group consisting of sodium carbonate, sodium bicarbonate, sodium sesquicarbonate, potassium carbonate, potassium bicarbonate, potassium sesquicarbonate, magnesium carbonate, sodium glycine carbonate, L-lysine carbonate, arginine carbonate, amorphous calcium carbonate, ammonium carbonate, ammonium bicarbonate and combinations thereof.

45. The process of claim 40, wherein the carrier in a) further comprises an acidic component, such as an organic acid, preferably as a powder.

46. The process of claim 45, wherein the acidic component selected from the group consisting of citric acid, tartaric acid, amalic acid, fumaric acid, adipic acid, lactic acid, succinic acid, disodium hydrogen phosphate, sodium dihydrogen phosphate, and combinations thereof.

47. The process of claim 40, which further includes a drying step, such as freeze-drying, vacuum drying, drying by means of a desiccant, or heating.

48. The process of claim 40, wherein the drying step is performed:
i) before the mixing step (ie one or more of the bacterial cells or carrier are dried); and/or
ii) after the mixing step (ie the mixture is dried); and/or
iii) after the optional compressing step (ie the powder, tablet or pellet is dried).

49. The process of claim 40, wherein the bacterial cells belonging to the genus Bifidobacterium are lactic acid producing and/or probiotic cells.

50. The process of claim 40, wherein the bacterial cells belongs to a species selected from the group consisting of: Bifidobacterium longum, Bifidobacterium bifidum, Bifidobacterium lactis, Bifidobacterium brevis, Bifidobacterium animalis, Bifidobacterium adolescentis, Bifidobacterium infantis.

51. The process of claim 40, wherein the bacterial cells belong to the species Bifidobacterium animalis, such as Bifidobacterium animalis subsp. lactis.

52. The process of claim 40, wherein the bacterial cells belong to a strain selected from the group consisting of: BB-12®, BB-02, Bifidobacterium animalis strain DSM15954, Bifidobacterium longum subsp. infantis strain DSM15953, Bifidobacterium longum subsp. longum strain DSM15955, and mutants or variants of any of these.

53. The process of claim 40, wherein the bacterial cells belong to the Bifidobacterium strain BB-12®.

54. The process of claim 40, wherein the bacterial cells belong to the Bifidobacterium strain BB-12® free.

55. The process of claim 40, where the carrier further comprises a component selected from the group consisting of: an inorganic acid or a salt thereof, an organic acid or a salt thereof, a carbohydrate, lactose, a sugar alcohol, soluble fibers and starch.

56. The process of claim 40, which further includes a coating step.

57. The process of claim 40, which further includes a packaging step, e.g. comprising placing the powder or compressed powder in a sealable container, e.g. made of aluminum and/or a polymer.

58. A composition obtainable by the process of claim 40.

59. A composition comprising bacterial cells belonging to the genus Bifidobacterium and a carrier comprising a salt of carbonic acid.

60. The composition of claim 59, wherein the bacterial cells belongs to a species selected from the group consisting of: Bifidobacterium longum, Bifidobacterium bifidum, Bifidobacterium lactis, Bifidobacterium brevis, Bifidobacterium animalis, Bifidobacterium adolescentis, Bifidobacterium infantis.

61. The composition of claim 59, wherein the bacterial cells belonging to the Bifidobacterium strain BB-12®.

62. The composition of claim 59, which comprises at least 10E5 CFU/mg, such as at least 10E7 CFU/mg or at least 10E9 CFU/mg, CFU being colony forming units of the bacterial cells belonging to the genus Bifidobacterium.

63. Use of the composition of claim 59, as a food or feed additive, a pharmaceutical, a dietary supplement, or a probiotic.

64. A food or feed product comprising a composition of claim 59.

65. The product of claim 59, which is a dairy product, such as milk or fermented milk.

66. The product of claim 64, which is a fruit juice.

67. A kit of parts, comprising a composition of claim 59 and a food product, e.g. a dairy product, such as milk or fermented milk, or a fruit juice.

68. The kit of claim 67, which comprises a container with a food product, and container with a composition comprising bacterial cells belonging to the genus Bifidobacterium and a carrier comprising a salt of carbonic acid.

69. Use of a salt of carbonic acid as a color stabilizer in a bacterial composition.

70. The use of claim 69, wherein the bacterial composition comprises bacterial cells belonging to the genus Bifidobacterium.

71. Use of a salt of carbonic acid for increasing the survival of the cells in a bacterial composition, wherein the bacterial composition comprises bacterial cells belonging to the genus Bifidobacterium.

72. Use of a salt of carbonic acid for increasing the shelf life of a bacterial composition, wherein the bacterial composition comprises bacterial cells belonging to the genus Bifidobacterium.

73. Use of a salt of carbonic acid for increasing the stability of a bacterial composition, wherein the bacterial composition comprises bacterial cells belonging to the genus Bifidobacterium.

74. The use of claim 73, wherein the bacterial cells belong to a species selected from the group consisting of: Bifidobacterium longum, Bifidobacterium bifidum, Bifidobacterium lactis, Bifidobacterium brevis, Bifidobacterium animalis, Bifidobacterium adolescentis, Bifidobacterium infantis.
